(19) United States
(12) Patent Application Publication
(10) Pub. No.: US 2023/0381449 A1
(43) Pub. Date: Nov. 30, 2023

(54) VAPOR ANNEALING TREATMENT TO IMPROVE COATING DURABILITY AND DRUG TRANSFER

(71) Applicant: Boston Scientific Scimed, Inc., Maple Grove, MN (US)

(72) Inventor: Ming Dang, Plymouth, MN (US)

(73) Assignee: Boston Scientific Scimed, Inc., Maple Grove, MN (US)

(21) Appl. No.: 18/202,153

(22) Filed: May 25, 2023

Related U.S. Application Data
(60) Provisional application No. 63/346,140, filed on May 26, 2022.

(51) Int. Cl. A61M 25/00; A61L 2/20

(52) U.S. Cl. CPC A61M 25/0009; A61L 2202/122; A61M 25/0045; A61L 2/20

(57) ABSTRACT

Medical devices and methods for making, using, and treating medical devices are disclosed. An example method for treating a medical device is disclosed. The method may include disposing an annealing fluid in a closed chamber, allowing the annealing fluid to reach a liquid-gas equilibrium within the chamber, and disposing a drug coated medical device within the closed chamber.

### CROSS-REFERENCE TO RELATED APPLICATIONS

This application claims the benefit of priority under 35 U.S.C. §119 of U.S. Provisional Application No. 63/346,140, filed May 26, 2022, the entire disclosure of which is hereby incorporated by reference.

### TECHNICAL FIELD

The present disclosure pertains to medical devices, and methods for manufacturing medical devices. More particularly, the present disclosure pertains to coated medical devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A method for treating a medical device is disclosed. The method comprises: disposing an annealing fluid in a closed chamber; allowing the annealing fluid to reach a liquid-gas equilibrium within the chamber; and disposing a drug coated medical device within the closed chamber.

Alternatively or additionally to any of the embodiments above, the annealing fluid comprises isopropyl alcohol.

Alternatively or additionally to any of the embodiments above, the closed container has a base and wherein disposing an annealing fluid in a closed chamber includes coating the base with the annealing fluid.

Alternatively or additionally to any of the embodiments above, coating the base with the annealing fluid includes coating the base to form a layer of the annealing fluid that is 1-10 millimeters thick.

Alternatively or additionally to any of the embodiments above, allowing the annealing fluid to reach a liquid-gas equilibrium within the chamber includes partially evaporating the annealing fluid.

Alternatively or additionally to any of the embodiments above, allowing the annealing fluid to reach a liquid-gas equilibrium within the chamber includes partially evaporating the annealing fluid for at least two hours.

Alternatively or additionally to any of the embodiments above, disposing a drug coated medical device within the closed chamber includes disposing the drug coated medical device within the closed chamber for at least two hours.

Alternatively or additionally to any of the embodiments above, the drug coated medical device includes a drug coating, and wherein disposing a drug coated medical device within the closed chamber includes vapor annealing the drug coating.

Alternatively or additionally to any of the embodiments above, the drug coating includes everolimus.

Alternatively or additionally to any of the embodiments above, the drug coated medical device includes a drug coated balloon.

Alternatively or additionally to any of the embodiments above, after disposing a drug coated medical device within the closed chamber, removing the drug coated medical device from the chamber and sterilizing the drug coated medical device.

A drug coated medical device is disclosed. The drug coated medical device comprises: an elongate catheter shaft having a distal end region; an expandable balloon coupled to the distal end region, the expandable balloon having an outer surface; a vapor annealed coating disposed along the outer surface; and wherein the coating includes everolimus.

Alternatively or additionally to any of the embodiments above, the vapor annealed coating is free of a polymer.

Alternatively or additionally to any of the embodiments above, the vapor annealed coating includes an excipient.

Alternatively or additionally to any of the embodiments above, the excipient includes acetyl tri-butyl citrate.

Alternatively or additionally to any of the embodiments above, the vapor annealed coating is vapor annealed with isopropyl alcohol.

A method for treating a medical device is disclosed. The method comprises: forming a vapor annealing chamber by disposing a layer of isopropyl alcohol within a container and allowing the layer of isopropyl alcohol to partially evaporate and reach a liquid-gas equilibrium; disposing an everolimus coated balloon within the vapor annealing chamber; vapor annealing the everolimus coated balloon within the vapor annealing chamber; and removing the vapor annealed everolimus coated balloon from the vapor annealing chamber.

Alternatively or additionally to any of the embodiments above, disposing an everolimus coated balloon within the vapor annealing chamber includes disposing the everolimus coated balloon within the vapor annealing chamber while the everolimus coated balloon is in a folded configuration.

Alternatively or additionally to any of the embodiments above, disposing an everolimus coated balloon within the vapor annealing chamber includes disposing the everolimus coated balloon within the vapor annealing chamber while the everolimus coated balloon is in an inflated configuration.

Alternatively or additionally to any of the embodiments above, after removing the vapor annealed everolimus coated balloon from the vapor annealing chamber, sterilizing the vapor annealed everolimus coated balloon.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic side view of an example medical device.

FIG. 2 is a schematic side view of an example medical device.

FIGS. 3-5 illustrate an example method.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Drug coated medical devices such as drug coated stents, drug coated balloons, and the like may be used to treat small vessel occlusions and/or vascular disease. Some example drugs that may be used with such devices includes paclitaxel, everolimus, sirolimus, etc. The application of the drug to the medical device may include applying a crystalline form of the drug to the surface of the medical devices. In at least some instances, the manufacturing process may include converting an amorphous form of the drug into a crystalline form. For example, crystalline everolimus (e.g., everolimus crystals) may be applied to the surface of a medical device and then converted to an amorphous form. It may be desirable to modify the coating morphology, for example to improve the durability of the coating and/or the drug transfer ability. Disclosed herein are processes for improving the durability of the coating and/or the drug transfer ability.

A number of coating processes are contemplated. For example, some example processes for converting an amorphous form of a drug into a crystalline form may generally include (a) preparing a suitable solvent, (b) preparing a nucleation initiator with the solvent, (c) combining/mixing the nucleation initiator with an amorphous form of a drug to form a drug precursor dispersion/suspension and (d) incubating the drug precursor dispersion/suspension to allow the amorphous form of the drug to convert to the crystalline form of the drug. The use of a suitable surfactant with the drug precursor dispersion/suspension may allow for the formation of drug crystals having a desirable morphology, allow for the formation of drug crystals having a desirable size and/or shape and/or aspect ratio, allow for the formation of coating suspensions with desirable stability, combinations thereof, and/or the like. Thus, at least some of the processes for converting an amorphous form of a drug into a crystalline form include (a) preparing a suitable solvent, (b) preparing a surfactant solution by combining/mixing the surfactant with the solvent, (c) preparing a nucleation initiator with the solvent, (d) combining/mixing the nucleation initiator with the surfactant solution and with an amorphous form of a drug to form a drug precursor dispersion/suspension and (e) incubating the drug precursor dispersion/suspension to allow the amorphous form of the drug to convert to the crystalline form of the drug.

In some embodiments, the drug may be a macrolide immunosuppressive (limus) drug. In some embodiments, the macrolide immunosuppressive drug is rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin 40-O-(2-Hydroxy)ethoxycarbonylmethyl-rapamycin, 40-O-(3-Hydroxy)propyl-rapamycin 40-O-(6-Hydroxy)hexyl-rapamycin 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin 40-O-(2-N-Imidazolylacetoxy)ethyl-rapamycin, 40-O-[2-(N-Methy-N′-piperazinyl)acetoxy]ethyl-rapamycin, 39-O-D esmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Toylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), or derivative, isomer, racemate, diastereoisomer, prodrug, hydrate, ester, or analog thereof. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof anti-coagulants; and growth factors.

As indicated herein, example processes for converting an amorphous form of a drug into a crystalline form include preparing a suitable solvent. When the solvent is a singular material, preparing a suitable solvent may be as simple as placing the solvent in a suitable container. When the solvent is a mixture of materials, preparing a suitable solvent may include combining or mixing the solvents.

In at least some instances, the solvent may include alcohols such as methanol, ethanol (EtOH), isopropanol (IPA), n-butanol, isobutyl alcohol or t-butyl alcohol; acetonitrile (ACN); ethers such as tetrahydrofuran (THF) isopropyl ether (IPE), diethyl ether (DEE); ketone solvents such as acetone, 2-butanone (MEK), or methyl isobutyl ketone (MIBK); halogenated solvents such as dichloromethane (DCM), monofluorobenzene (MFB), α,α,α-trifluorotoluene (TFT), nitromethane (NM), ethyl trifluoroacetate (ETFA); aliphatic hydrocarbons such as hexane, heptane, or the like; aromatic hydrocarbons, such as toluene or xylenes; and ester solvents such as ethyl acetate. Mixed solvents such as ethyl acetate/heptane, acetone/water, IPA/water, IPA/THF, methanol/water, IPA/heptane, or THF/heptane can also be used, for example.

When mixtures of solvents are utilized, the mixture may have a suitable ratio of each material. For example, some example solvents may include a mixture of ethyl acetate and heptane. The ratio of ethyl acetate to heptane may be in the range of about 1:4 to 1:30, or about 1:4 to 1:20, or about 1:20. Other mixtures and/or ratios are contemplated. Thus, in some instances the process for converting an amorphous form of a drug into a crystalline form may include preparing a solvent, for example by mixing ethyl acetate with heptane.

The process for converting an amorphous form of a drug into a crystalline form may include preparing a surfactant solution (e.g., preparing a surfactant solution with the solvent). In at least some instances, the surfactant may include TWEEN 20™ (e.g., polysorbate 20, polyoxyethylene (20) sorbitan monooleate, or PEG (20) sorbitan monooleate), TWEEN 80™ (e.g., polysorb ate 80, polyoxyethylene (80) sorbitan monooleate, or PEG (80) sorbitan monooleate), SPAN™ 80, SPAN™ 20, TRITON™ X-100, TRITON™ 400, a non-ionic surfactant, and/or the like. The surfactant solution may have a suitable concentration. For example, the concentration of the surfactant solution may be about 0.01-1% (by weight), about 0.05-0.5% (by weight), or about 0.1% (by weight).

The process for converting an amorphous form of a drug into a crystalline form may include preparing a nucleation initiator (e.g., preparing a nucleation initiator with the solvent). This process or step may also be termed “seeding”. In at least some instances, the nucleation initiator may include a crystalline form of the drug suspended in a suitable solvent (e.g., which may or may not be the same solvent that is used to dissolve the amorphous form of the drug). When the drug utilized is everolimus, the nucleation initiator may include a suitable quantity of crystalline everolimus in a suitable solvent. The concentration of the of the crystalline everolimus in the solvent may be in the ration of about 0.1-10%, or about 0.1-2%, or about 0.5%. The crystalline everolimus may include everolimus microcrystals formed as described herein. Alternatively, the crystalline everolimus may include everolimus crystals having a different morphology.

The process for converting an amorphous form of a drug into a crystalline form may include mixing and/or combining the nucleation initiator with the surfactant solution and with an amorphous form of a drug to form a drug precursor dispersion/suspension. The amount of the amorphous form of the drug added may vary. For example, about 0.5-100 milligrams of everolimus may be added/dispersed per milliliter (e.g., per milliliter of solvent), or about 1-50 milligrams of everolimus may be added/dispersed per milliliter, or about 10-30 milligrams of everolimus may be added/dispersed per milliliter, or about 20 milligrams of everolimus may be dissolved per milliliter. In at least some instances, the drug dispersion solution may be termed and/or resemble a slurry.

The drug precursor dispersion/suspension may be incubated so that the amorphous form of the drug may converts to the crystalline form. In some instances, incubation may occur over a suitable time period on the order of a number of hours to a number of days. For example, incubation may occur over 24 hours. In some of these and in other instances, incubation may include a high-temperature or warm-temperature incubating step at a suitable temperature (e.g., at about 20° C. to 80° C., or at about 40° C. to or at about 50° C.). In some instances, the high-temperature incubating may include agitating the drug precursor dispersion (e.g., using a suitable device such as an orbital shaker). However, in other instances, the high-temperature incubating step is free from agitating/agitation. The high-temperature incubating step may occur over a suitable time period on the order of a number of hours to a number of days. For example, incubation may occur over approximately two days. In some of these and in other instances, incubation may also include a low-temperature or cool-temperature incubating step at a suitable temperature (e.g., at about −10° C. to 10° C., or at about 0° C. to 5° C., or at about 4° C.). The low-temperature incubating step may occur over a suitable time period on the order of a number of hours to a number of days. For example, incubation may occur over several days.

In some instances, the process for converting an amorphous form of a drug into a crystalline form may include one or more of (a) filtering the crystalline form of the drug, (b) washing the crystalline form of the drug, and (c) drying the crystalline form of the drug. For example, some processes are contemplated that include filtering, washing, and drying the crystalline form of the drug. When doing so, the solvent, the surfactant, or both may be essentially completely removed from the drug crystals.

The crystalline form of the drug (e.g., the crystalline form of everolimus) formed by this process may result in crystals with a morphology, size, and shape that allow the formed crystals to be described as being microcrystals. In particular, the use of a surfactant in the crystallization process, along with a suitable solvent mixture, mixed at a suitable ratio, results in the formation of microcrystals. For the purposes of this disclosure, microcrystals may be understood to be crystals that could be described as relatively flat, thin sheets. Some example dimensions may include microcrystal having a width less than about 3 micrometers (e.g., having a non-zero width that is less than about 3 micrometers), a thickness less than about 1 micrometer (e.g., having a non-zero thickness that is less than about 1 micrometer), and a length less than about 10 micrometers (e.g., having a non-zero length that is less than about 10 micrometers). Microcrystals may be desirable for a number of reasons. For example, in some crystallization processes that form “larger”, rod-like, or “non-micro” crystals, the resultant crystals can rapidly settle when suspended in a coating material/dispersion. This may make it more challenging to apply the drug crystals to a medical device. The microcrystals formed by the process described herein can be, for example, suspended in a coating material/dispersion and remain in suspension for an extended period of time (e.g., on the order of months or longer).

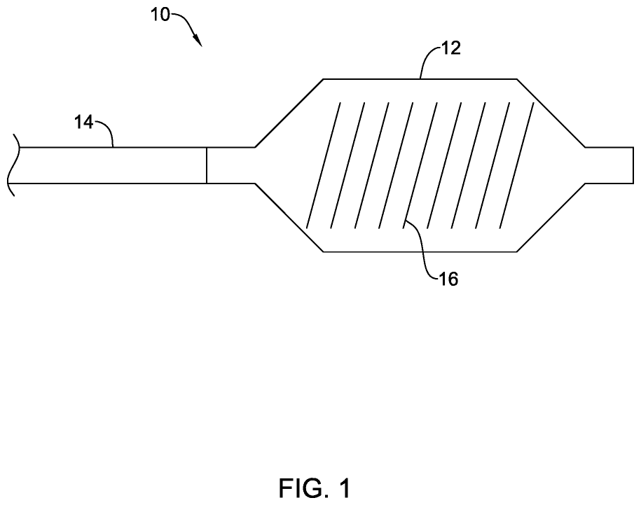

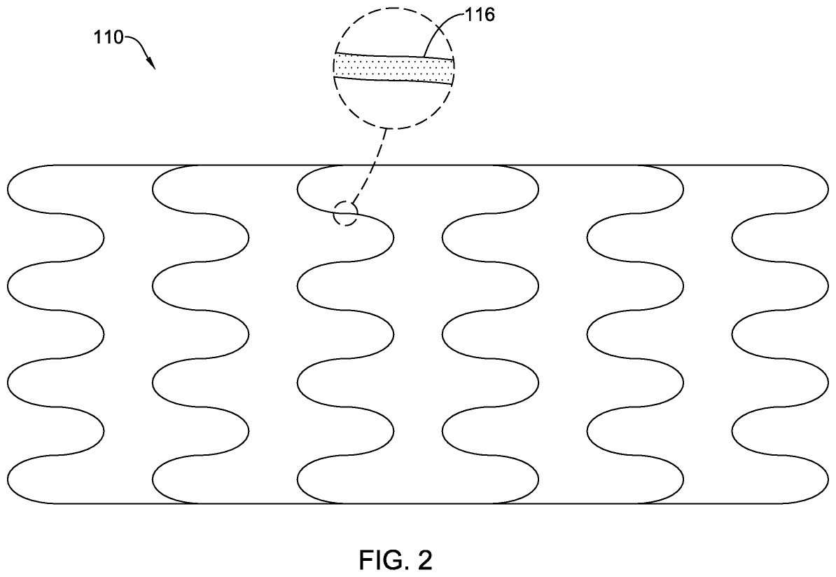

In some instances, the microcrystals formed by the process disclosed herein may be suspend in a suitable coating material/dispersion. This may include the addition of a suitable excipient. An example excipient may include acetyl tri-butyl citrate (ATBC). In some instances, the drug microcrystals may be mixed with the excipient at a ratio of about 20:80 to about 90:10 or at a ratio of about 80:20. The coating material/dispersion may then be applied to a medical device using a suitable process. For example, the coating material/dispersion may then be applied to a medical device by dip coating, roll coating, via a syringe, and/or the like. Some example drug coated medical devices, shown schematically, are shown in FIGS. 1-2. For example, FIG. 1 illustrates a drug coated balloon device 10 including a balloon 12 coupled to a catheter shaft 14. A drug coating 16 (e.g., crystalline everolimus including everolimus microcrystals formed as disclosed herein) may be disposed along the balloon 12. FIG. 2 illustrates a drug coated stent 110. A drug coating 116 (e.g., crystalline everolimus including everolimus microcrystals formed as disclosed herein) may be disposed along the stent 110.

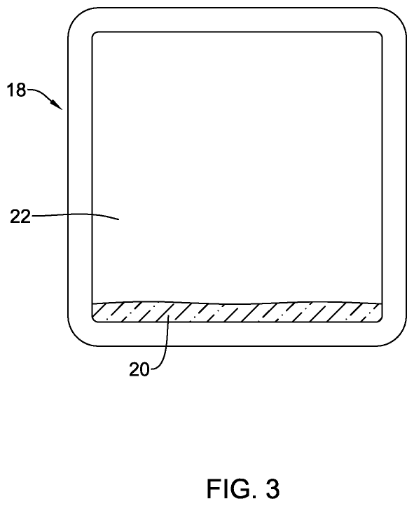

Once coated, the drug coated balloon device 10 and/or the drug coated stent 110 can be treated and/or otherwise subjected to further processing in order to modify the coating morphology (e.g., in order to improve coating durability and/or drug transfer ability). FIGS. 3-5 schematically depict an example process for treating/modifying the coating morphology. The example process is a vapor annealing process where the drug coated balloon device 10 and/or the drug coated stent 110 (e.g., in the example FIGS. 3-5, the drug coated balloon device 10 is shown; however other devices including the drug coated stent 110 can undergo similar processes).

An example vapor annealing process may include forming a vapor annealing chamber by disposing an annealing fluid 20 (e.g., which may take the form of a volatile substance such as isopropyl alcohol (IPA)) in a chamber 18. In FIG. 3, the chamber 18 is depicted schematically with a layer of the annealing fluid 20 disposed along the bottom of the chamber 18. Depending on the size of the chamber, the layer of the annealing fluid 20 may be about 1-10 mm thick or more, or about 1-5 mm thick or more, or about 1.5-3 mm thick or more, or about 2 mm thick or more. Initially, the remaining open space 22 of the chamber 18 may only be filled with air or a controlled atmosphere.

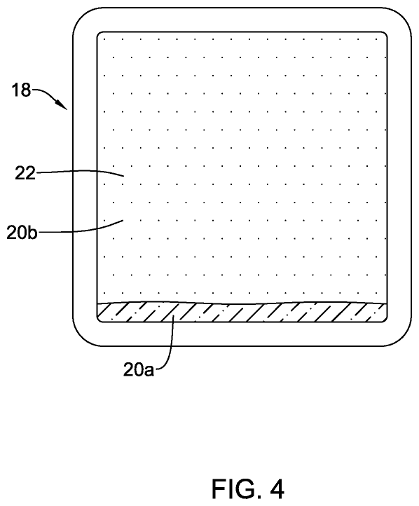

The annealing fluid 20 may be allowed to at least partially evaporate within the chamber 18 (e.g., while the chamber 18 is closed or sealed) until the annealing fluid 20 reaches a liquid-gas equilibrium as depicted in FIG. 4. For example, the chamber 18 depicted in FIG. 4 (e.g., a vapor annealing chamber 18) may include both a liquid portion 20a of the annealing fluid 20 and a vapor portion 20b of the annealing fluid 20. The amount of time required to reach a liquid-gas equilibrium may vary and can include about 1-10 hours or more, or about 1-5 hours or more, or about 1.5-3 hours or more, or about 2 hours or more.

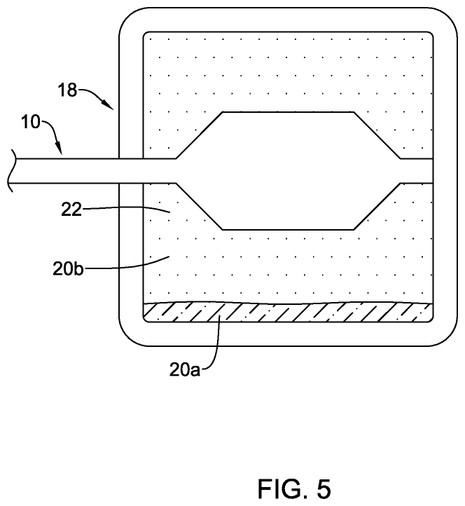

When the annealing fluid 20 reaches a suitable equilibrium, a medical device with a coating (e.g., the drug coated balloon device 10) may be disposed in the vapor annealing chamber 18 (e.g., the closed vapor annealing chamber 18) as shown in FIG. 5. This may include disposing the drug coated balloon device 10 within the vapor annealing chamber 18 while in a folded configuration, an unfolded configuration, a partially inflated configuration, an inflated configuration, or another suitable configuration. In at least some instances, the drug coated balloon device 10 may be disposed in vapor annealing chamber 18 in a manner that keeps the drug coated balloon device 10 from contacting the layer of the annealing fluid 20 (e.g., the liquid portion 20a of the annealing fluid 20). The drug coated balloon device 10 may be disposed within the vapor annealing chamber 18 for a suitable time period. For example, the drug coated balloon device 10 may be disposed within the vapor annealing chamber 18 for about 1-10 hours or more, or about 1-5 hours or more, or about 1.5-3 hours or more, or about 2 hours or more.

After undergoing the vapor annealing process, the drug coated balloon device 10 can be removed from the vapor annealing chamber 18. In some instances, the drug coated balloon device 10 (e.g., the now vapor annealed drug coated balloon device 10) can undergo additional processes such as folding, disposing the drug coated balloon device 10 into a balloon protector, sterilization (e.g., ethylene oxide sterilization), packaging, combinations thereof, and/or the like.

The drug coated balloon device 10 (e.g., the now vapor annealed drug coated balloon device 10) may have a number of desirable properties. For example, drug coated medical devices that are not vapor annealed may have a drug layer with a dry, powder-like morphology. In contrast, the drug coated balloon device 10 (e.g., the now vapor annealed drug coated balloon device 10) may have a drug layer with a wet-like, smooth morphology. The drug layer may also have an improved durability and/or an improved drug transfer ability (e.g., when compared to drug layers that are not vapor annealed).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for treating a medical device, the method comprising:
disposing an annealing fluid in a closed chamber;
allowing the annealing fluid to reach a liquid-gas equilibrium within the chamber; and
disposing a drug coated medical device within the closed chamber.

2. The method of claim 1, wherein the annealing fluid comprises isopropyl alcohol.

3. The method of claim 1, wherein the closed container has a base and wherein disposing an annealing fluid in a closed chamber includes coating the base with the annealing fluid.

4. The method of claim 3, wherein coating the base with the annealing fluid includes coating the base to form a layer of the annealing fluid that is 1-10 millimeters thick.

5. The method of claim 1, wherein allowing the annealing fluid to reach a liquid-gas equilibrium within the chamber includes partially evaporating the annealing fluid.

6. The method of claim 1, wherein allowing the annealing fluid to reach a liquid-gas equilibrium within the chamber includes partially evaporating the annealing fluid for at least two hours.

7. The method of claim 1, wherein disposing a drug coated medical device within the closed chamber includes disposing the drug coated medical device within the closed chamber for at least two hours.

8. The method of claim 1, wherein the drug coated medical device includes a drug coating, and wherein disposing a drug coated medical device within the closed chamber includes vapor annealing the drug coating.

9. The method of claim 8, wherein the drug coating includes everolimus.

10. The method of claim 1, wherein the drug coated medical device includes a drug coated balloon.

11. The method of claim 1, after disposing a drug coated medical device within the closed chamber, removing the drug coated medical device from the chamber and sterilizing the drug coated medical device.

12. A drug coated medical device, comprising:
an elongate catheter shaft having a distal end region;
an expandable balloon coupled to the distal end region, the expandable balloon having an outer surface;
a vapor annealed coating disposed along the outer surface; and
wherein the coating includes everolimus.

13. The drug coated medical device of claim 12, wherein the vapor annealed coating is free of a polymer.

14. The drug coated medical device of claim 12, wherein the vapor annealed coating includes an excipient.

15. The drug coated medical device of claim 14, wherein the excipient includes acetyl tri-butyl citrate.

16. The drug coated medical device of claim 12, wherein the vapor annealed coating is vapor annealed with isopropyl alcohol.

17. A method for treating a medical device, the method comprising:
forming a vapor annealing chamber by disposing a layer of isopropyl alcohol within a container and allowing the layer of isopropyl alcohol to partially evaporate and reach a liquid-gas equilibrium;
disposing an everolimus coated balloon within the vapor annealing chamber;
vapor annealing the everolimus coated balloon within the vapor annealing chamber; and
removing the vapor annealed everolimus coated balloon from the vapor annealing chamber.

18. The method of claim 17, wherein disposing an everolimus coated balloon within the vapor annealing chamber includes disposing the everolimus coated balloon within the vapor annealing chamber while the everolimus coated balloon is in a folded configuration.

19. The method of claim 17, wherein disposing an everolimus coated balloon within the vapor annealing chamber includes disposing the everolimus coated balloon within the vapor annealing chamber while the everolimus coated balloon is in an inflated configuration.

20. The method of claim 17, after removing the vapor annealed everolimus coated balloon from the vapor annealing chamber, sterilizing the vapor annealed everolimus coated balloon.
